(19) United States
(12) Patent Application Publication
(10) Pub. No.: US 2010/0168631 A1
(43) Pub. Date: Jul. 1, 2010

(54) ANKLE PROTECTOR

(76) Inventors: Harald Scheffer, Merzig (DE); Robert Steed, Essex (GB)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 12/667,048

(22) PCT Filed: Jun. 25, 2008
(86) PCT No.: PCT/GB2008/050489

§ 371 Date: Jan. 12, 2010

(30) Foreign Application Priority Data
Jun. 29, 2007 (GB) 0712551.1

(51) Int. Cl. A61F 5/00

(52) U.S. Cl. 602/27

(57) ABSTRACT

An ankle protector (10) comprises an elastic webbing element (11), formed with a tubular construction thereby to surround and embrace a user's ankle region. The webbing element (11) is defined by upper and lower apertures (12, 13) to enable a user's foot (21) to pass therethrough when putting the ankle protector (10) on. Each of said upper and lower apertures (12, 13) is formed by overlock-stitching (22) an edge of the elastic webbing (11) material, such that said apertures (12, 13) have no perceptible ribs. A cushioning element (17) is attached to the webbing element (11) and is adapted to cover and protect a user's Achilles tendon. The cushioning element (17) protrudes inwardly relative to the circumference of the tubular webbing element (11).

This invention relates to an ankle protector. In particular, the invention relates to an ankle protector to be worn over a user's ankle region during the performance of contact sports.

The present invention has been developed for use in contact sports in general, and association football (soccer) in particular. The invention will therefore be described herein with particular emphasis on the sport of football, though it should be understood that the ankle protector of the present invention is equally suitable for use in other contact sports such as rugby, American football and hockey—and indeed for use in non-contact sports to give protection against accidental contact with other players or equipment.

The use of protective apparel by footballers has become widespread at both professional and amateur level. Aside from the stresses and strains applied to an athlete's body by the performance of any physical sport, the most likely cause of an injury occurring during a football game is by the accidental, but often inevitable, contact between opposing players' feet and legs during the execution of a tackle. For this reason, most players wear shin pads to absorb such impacts.

Many commercially available shin pads now incorporate an ankle protector section stitched onto the main part of the shin pad, and adapted to be secured under the user's foot by means of an elastic loop or stirrup. However, such shin pad and ankle protector combinations tend to be cumbersome and uncomfortable for the user, and can restrict the range of motion of the ankle joint.

Separate ankle protectors are also available, which avoid the motion restriction problems associated with combined shin pad and ankle protection systems. However, existing ankle protectors suffer from shortcomings which at best make them uncomfortable for the user to wear, and at worst leave the user prone to injury. One such shortcoming derives from the typical construction of the ankle protector from conventional elasticated fabric material. In order to prevent the fabric material unravelling, the protectors are generally formed with a thick hem or seam—generally referred to herein as a “rib”—around the upper and/or lower apertures through which the user's foot passes when putting the ankle protector on. As the protector is designed to be worn tightly against the user's skin under a sock, these ribs can press into the skin of the user's foot and lower leg during a football game, causing severe discomfort—or worse, if a poorly timed tackle happens to impact at the exact location of a rib.

Another shortcoming of existing ankle protectors is that they tend to concentrate on providing compressive support to the ankle joint, so as to protect against ankle twists, strains and sprains. Whilst this kind of protection is important, it is at least equally as important for a footballer to be protected against impact during tackles. Existing ankle protectors tend to overlook this aspect, with the user's lateral and medial malleoli often left effectively unprotected, and protection for the Achilles tendon usually neglected altogether.

The present invention seeks to address the above issues by providing an improved ankle protector, developed particularly for use by footballers, which provides improved levels of comfort to the user by dispensing with the need for cumbersome ribs around the protector's apertures, without compromising the level of protection afforded to the user of the integrity of the protector. In preferred embodiments, the ankle protector also addresses the issues of providing enhanced impact protection to the user's malleoli and Achilles tendon.

According to the present invention there is provided an ankle protector comprising:
an elastic webbing element, formed with a tubular construction thereby to surround and embrace a user's ankle region, said webbing element being defined by upper and lower apertures to enable a user's foot to pass therethrough when putting the ankle protector on, and wherein each of said upper and lower apertures is formed by overlock-stitching an edge of the elastic webbing material, such that said apertures have no perceptible ribs; and
a cushioning element attached to the webbing element and adapted to cover and protect a user's Achilles tendon, said cushioning element protruding inwardly relative to the circumference of the tubular webbing element.

The term “perceptible” is used in this context to mean that no discomfort will be caused to the user by the edges of the ankle protector in contact with his or her skin, when the ankle protector is in use, for example during a game of football. Ideally, the user will be substantially unable to feel the edges of the ankle protector through his or her skin, during use.

The elastic webbing element is preferably formed from a substantially non-absorbent, impermeable material. This ensures that the material does not absorb moisture—either from atmospheric humidity or precipitation, or from the user's perspiration—so that it does not swell or slip during use.

In a preferred embodiment of the present invention, the elastic webbing element is formed from a material comprising nylon and neoprene. Most preferably, the material comprises substantially 61% nylon and substantially 39% neoprene.

The cushioning element addresses the issue of providing enhanced support and impact protection. The cushioning element preferably comprises a pair of elongate cushioning members, arranged substantially parallel to one another and adapted to embrace either side of the tendon.

The tubular elastic webbing element may desirably be formed with a continuous, seamless construction. However, in practice such a construction may not always be achievable. Where a seam is present, it may be located at the front of the ankle protector, diametrically opposite the cushioning element. Alternatively, a seam may be located between the parallel elongate cushioning members so as to minimise or eliminate the user's perception of the seam. Where a seam is present, it is preferred that the webbing element be formed with only a single seam, said single seam being located in either one or the other of the above described locations.

The cushioning element is preferably formed from expanded polyethylene (EPE) foam, and may conveniently be stitched into or onto the elastic webbing element. In preferred embodiments of the present invention, the cushioning element protrudes both inwardly and outwardly relative to the circumference of the tubular webbing element. The inwardly-directed protrusion is adapted to embrace either side of the user's Achilles tendon as described above, whilst the outwardly-directed protrusion serves to cushion the Achilles tendon against impact. The preferred thickness of the cushioning element, to provide optimum protection without compromising comfort or mobility has been found to be substantially 5 mm.

In order to provide additional impact protection to the user, the ankle protector preferably further comprises a pair of disc-shaped shield elements located within the webbing element and adapted to cover and protect a user's lateral and medial malleoli. The shield elements will generally be formed from plastics material, preferably polyethylene (PE), and stitched into the elastic webbing element.

The ankle protector of the present invention preferably further comprises an elastic stirrup attached to the webbing element and adapted to embrace the underside of a user's foot, thereby to retain the ankle protector in position on the user's ankle. In order further to enhance the comfort provided to the user, it is preferred that the stirrup should be formed from a single piece of elastic material, without any seams. It is particularly preferred that no seams are located adjacent the underside of the user's foot.

The present invention has been described thus far in terms of a single ankle protector. In reality of course, the user will have need for a pair of such ankle protectors, one to be worn on each ankle. The scope of the present invention therefore extends to encompass an ankle protector kit comprising a pair of ankle protectors as hereinbefore described, one member of said pair being shaped so as in use to embrace a user's left ankle, and the other member of said pair being shaped so as in use to embrace a user's right ankle.

In order that the present invention may be better understood, a preferred embodiment thereof will now be described, though only by way of example, with reference to the accompanying drawings, in which:

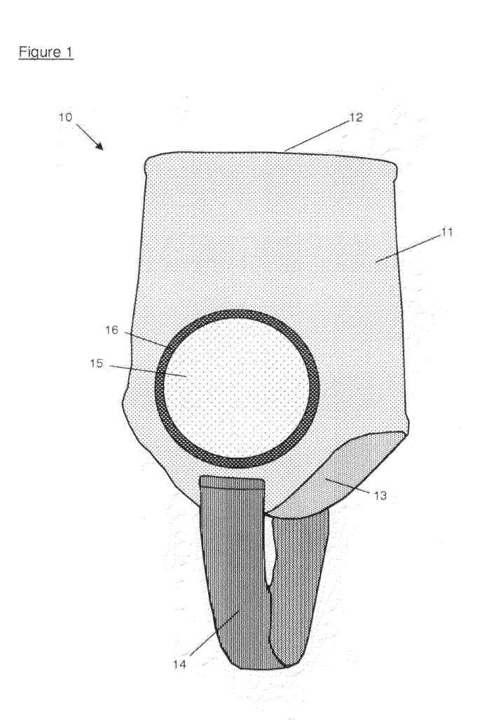

FIG. 1 shows a perspective front/side view of an ankle protector, according to the present invention;

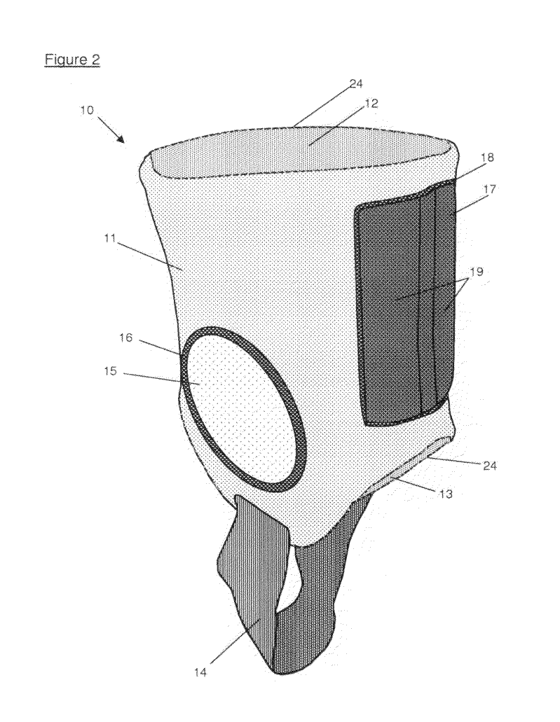

FIG. 2 shows a perspective rear/side view of the ankle protector of FIG. 1;

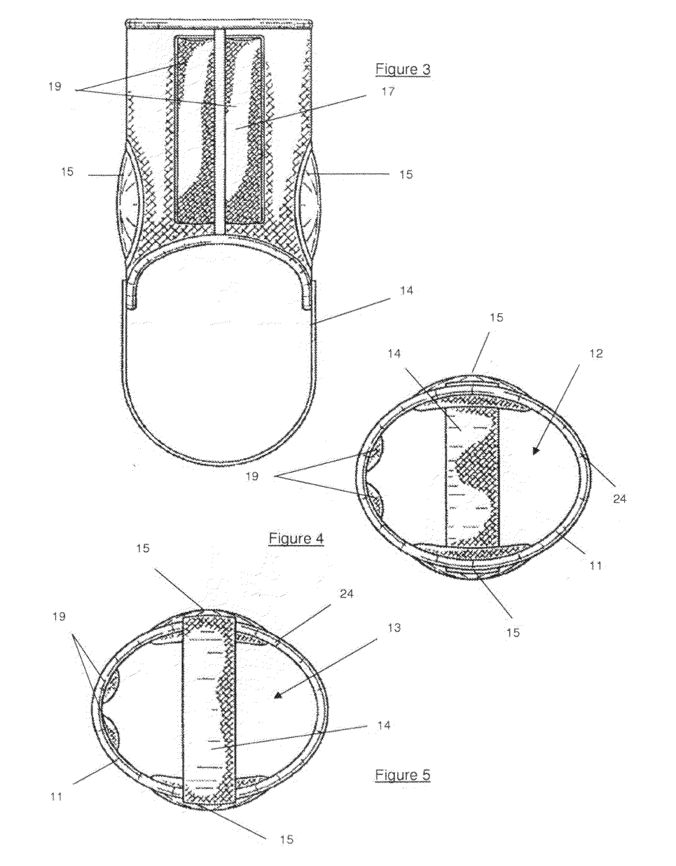

FIG. 3 shows a rear view of the ankle protector of FIGS. 1 and 2;

FIG. 4 shows a plan view of the ankle protector of FIGS. 1 to 3;

FIG. 5 shows a view of the underside of the ankle protector of FIGS. 1 to 4; and

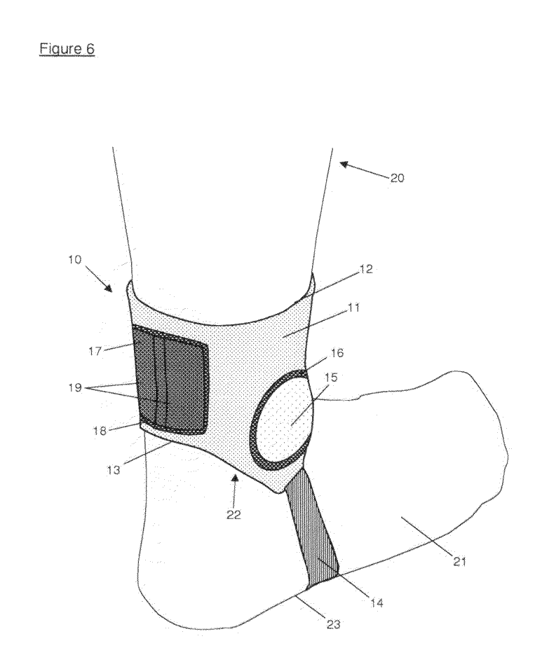

FIG. 6 shows an alternative perspective rear/side view of the ankle protector of FIGS. 1 to 5 in use when worn on a user's ankle region.

Referring first to FIG. 1, there is shown an ankle protector, generally indicated 10. The ankle protector 10 comprises an elastic webbing element 11, formed with a tubular construction from a single piece of elastic webbing material. The webbing element 11 is defined by an upper aperture 12 and a lower aperture 13, each adapted to allow a user's foot and ankle to pass therethrough when putting on the ankle protector 11, as will be described in more detail below with reference to FIG. 6.

The upper and lower apertures 12, 13 are each formed by applying overlock stitching to an edge of the elastic webbing material 11, so that the apertures 12, 13 have no perceptible ribs.

The ankle protector 10 is provided with an elasticated stirrup 14 bridging the lower aperture 13. The stirrup 14 is designed to pass under the user's foot and to retain the ankle protector 10 in position during use, and is formed from a single piece of elastic material, so that no seams are brought into contact with the underside of the user's foot.

The ankle protector 10 is further provided with disc-shaped shield elements 15, stitched into or onto the elastic webbing material 11 by a ring of heavy duty zig-zag stitching 16. As shown in FIG. 3, the protector 10 is provided with a pair of shield elements 15, one disposed at either side of the protector 10, such that in use one shield element 15 is arranged to overlie and protect the user's lateral malleolus and the other shield element 15 is arranged to overlie and protect the user's medial malleolus, as will be described in more detail below with reference to FIG. 6.

Referring now to FIGS. 2 and 3, it can now be seen that the ankle protector 10 is further provided with a cushioning element 17, stitched into or onto the elastic webbing material 11 by a line of heavy duty stitching 18. The cushioning element 17 is arranged so as in use to overlie and protect the user's Achilles tendon area.

The cushioning element 17 comprises a pair of elongate cushioning members 19, adapted to embrace and cushion either side of a user's Achilles tendon. As shown in FIGS. 4 and 5, the cushioning members 19 protrude inwardly relative to the circumference of the tubular webbing element 11 in order to achieve this; and, as shown in FIGS. 2 and 3, also protrude outwardly relative to the circumference of the tubular webbing element 11 in order to cushion the user's Achilles tendon against impact. FIG. 4 provides a clearer view of the upper aperture 12 through which the user must insert his or her foot and ankle in order to put on the ankle protector 10 of the present invention, said aperture 12 and its corresponding lower aperture 13 being formed by applying overlock stitching 24 to an edge of the elastic webbing material 11, so that the apertures 12, 13 have no perceptible ribs. FIG. 5 shows a clearer view of the lower aperture 13 and the seamless stirrup 14 for retaining the ankle protector 10 on a user's foot.

Referring now to FIG. 6, this shows the ankle protector 10 being worn by a user, generally indicated 20. In order to put on the ankle protector 10 of the present invention, the user 20 inserts his or her foot 21 into the upper aperture 12, through the tubular elastic webbing element 11, out through the lower aperture 13, and into the stirrup 14, thus arriving at the position shown in FIG. 6. The ankle protector 10 is now in position on the user's ankle region, generally indicated 22, extending from immediately below the lateral and medial malleoli to immediately above the ankle joint. The stirrup 14 now passes under the sole 23 of the user's foot 21, to retain the ankle protector 10 in place.

As can be seen from FIG. 6, the ankle protector 10 provides protection to the user by virtue of the shield elements 15 which overlie the user's lateral and medial malleoli, and the cushioning element 17 which overlies the user's Achilles tendon. Additionally, the elastic webbing element 11 provides compressive support to the whole ankle region 22. The edges of the elastic webbing element 11 at the upper aperture 12 and lower aperture 13 lie against the skin of the user 20—however, as the overlock stitching 24 enables said edges to be substantially free from any perceptible ribs, the user's comfort levels are greatly increased and the risk of injury is greatly reduced.

1. An ankle protector comprising:
an elastic webbing element, formed with a tubular construction thereby to surround and embrace a user's ankle region, said webbing element being defined by upper and lower apertures to enable a user's foot to pass therethrough when putting the ankle protector on, and wherein each of said upper and lower apertures is formed by overlock-stitching an edge of the elastic webbing material, such that said apertures have no perceptible ribs;
a cushioning element attached to the webbing element and adapted to cover and protect a user's Achilles tendon, said cushioning element protruding inwardly relative to a circumference of the tubular webbing element; and
a pair of disc-shaped shield elements located within the webbing element and adapted to cover and protect a user's lateral and medial malleoli.

2. The ankle protector as claimed in claim 1, wherein, when the ankle protector is in use, the elastic webbing element extends from immediately below the user's malleoli to immediately above the user's ankle joint.

3. The ankle protector as claimed in claim 1, wherein the elastic webbing element is formed from a substantially non-absorbent, impermeable material.

4. The ankle protector as claimed in claim 1, wherein the elastic webbing element is formed from a material comprising nylon and neoprene.

5. The ankle protector as claimed in claim 1, wherein the elastic webbing element is formed from a material comprising substantially 61% nylon and substantially 39% neoprene.

6. The ankle protector as claimed in claim 1, wherein the cushioning element is stitched into or onto the elastic webbing element.

7. The ankle protector as claimed in claim 1, wherein the cushioning element further protrudes outwardly relative to the circumference of said tubular webbing element.

8. The ankle protector as claimed in claim 1, wherein the cushioning element comprises a pair of elongate cushioning members, arranged substantially parallel to one another and adapted to embrace either side of a user's Achilles tendon.

9. The ankle protector as claimed in claim 8, wherein the tubular webbing element is formed with a seam located between the parallel elongate cushioning members.

10. The ankle protector as claimed in claim 1, wherein the tubular webbing element is formed with a seam located diametrically opposite the cushioning element.

11. The ankle protector as claimed in claim 10, wherein said seam is the only seam formed in the webbing element.

12. The The ankle protector as claimed in claim 1, wherein the tubular webbing element is formed from a single continuous piece of material having no seams.

13. The ankle protector as claimed in claim 1, wherein the cushioning element is formed from expanded polyethylene (EPE) foam.

14. The ankle protector as claimed in claim 1, wherein the cushioning element has a thickness of substantially 5 mm.

15. (canceled)

16. The ankle protector as claimed in claim 1, wherein the shield elements are formed from polyethylene (PE).

17. The ankle protector as claimed in claim 1, wherein the shield elements are stitched into the elastic webbing element.

18. The ankle protector as claimed in claim 1, further comprising an elastic stirrup attached to the webbing element and adapted to embrace the underside of a user's foot, thereby to retain the ankle protector in position on the user's ankle.

19. The ankle protector as claimed in claim 18, wherein the stirrup is formed from a single piece of elastic material and comprises no seams.

20. An ankle protector kit comprising a pair of the ankle protectors of claim 1, one member of said pair being shaped so as in use to embrace a user's left ankle, and the other member of said pair being shaped so as in use to embrace a user's right ankle.

21. The ankle protector as claimed in claim 9, wherein said seam is the only seam formed in the webbing element.
